(19) United States
(12) Patent Application Publication
(10) Pub. No.: US 2004/0092504 A1
(43) Pub. Date: May 13, 2004

(54) Definitive medications for treating fibromyalgia

(76) Inventor: Anuthep Benja-Athon, New York, NY (US)

Correspondence Address: ANUTHEP BENJA-ATHON, M.D., Rehabilitation Medicine, Acupuncture Physical Therapy, 210 East 36th Street, New York, NY, 10016, US

(21) Appl. No.: 10/290,786

(22) Filed: Nov. 12, 2002

(51) Int. Cl. A61K 31/55; A61K 31/4168; A61K 31/19

(52) U.S. Cl. 514/217; 514/557; 514/389

(57) ABSTRACT

Base on the anatomy and neurophysiology described in the Neurophysiologic Basis of Idiopathic Diseases, the categories of oral and parenteral medications can be used to manage and treat fibromyalgia and related diseases, disorders, syndromes and sequelae in a human. The target neurons involve in the genesis and perpetuation of fibromyalgia and related syndromes, diseases and disorders and sequelae in the peripheral nervous system and central nervous system are affected and modulated by the anticonvulsants, antidepressants and opioids.

### FIELD OF INVENTION

The categories of oral and parenteral medications for the treatment of fibromyalgia and related diseases and disorders.

### BACKGROUND OF THE INVENTION

This applicant filed a patent application entitled the Neurophysiologic Basis of Idiopathic Diseases on Oct. 21, 2002 as a continuation-in-part application of U.S. Pat. No. 5,861,015 granted to this applicant on Jan. 19, 1999 entitled Modulation of the Nervous Systems for Treatment of Pain and Related Disorders. In said application and patent, the applicant has elucidated the anatomy and neurophysiology of the part of the nervous systems including the neural circuitries in the peripheral and central nervous systems which are involved in the genesis and perpetuation of various diseases and disorders labeled with the so-called “idiopathic, unknown or unclear etiology, pathogenesis or mechanism”. Said diseases and disorders are such as, but not limited to, fibromyalgia and related disorders, illnesses, syndromes and sequelae.

Except for U.S. Pat. No. 5,861,015 granted to this applicant entitled Modulation of the Nervous Systems for Treatment of Pain and Related Disorders using therapeutic electricity, there is neither known nor established oral and parenteral medications for the treatment and modulation of said diseases and disorders. The reason is obvious as stated supra.

The first objective of the present invention is the discovery of the categories of oral and parenteral medications which are effective in managing and treating fibromyalgia and related diseases and disorders based on the applicant's theory in said application and patent.

The second objective of the present invention is the elucidation of the mechanism of said categories of oral and parenteral medications which are effective in managing and treating fibromyalgia and related diseases and disorders based on said theory.

The third objective leads to the specific and objective uses of said oral and parenteral medications in the management and treatment of said diseases and disorders.

The fourth objective is to lead to and support further researches and medical advancements in the discovery the preventions, effective treatments and cures for many common diseases and disorders afflicting humans.

The social, health and economic implications of said objectives are immense.

### SUMMARY OF THE INVENTION

Before said US patent and application, many common and disabling diseases and disorders were labeled with “idiopathic, unknown etiology, cause, pathogenesis and biomechanism”. As a result, the proper use of the oral and parenteral medications for the management, treatment said diseases and disorders was not known prior to this application. A significant advancement in medicine was prevented.

Therefore, the categories of oral and parenteral medications comprise the anticonvulsants, antidepressants and opioids can be specifically used to manage and treat fibromyalgia and related diseases, disorders, syndromes and to prevent their sequelae in a human. The target neurons and the neural circuitries involve in the genesis and perpetuation of fibromyalgia and related diseases, disorders, syndromes and sequelae in the peripheral nervous system and central nervous system as described in said applicaion are affected and modulated by said medications.

### PREFERRED EMBODIMENTS OF THE INVENTION

Although said Neurophysiologic Basis of Idiopathic Diseases is not a part of this patent application, however, it is necessary to refer to said application in order to understand the target neurons, neural circuitries, sites of the effects and mechanism of the oral and parenteral medications stated herein.

Therefore, according to the anatomy and neurophysiology described in said Neurophysiologic Basis of Idiopathic Diseases, the small, medium and large unmyelinated and myelinated afferent neurons, postganglionic sympathetic neurons, preganglionic sympathetic neurons, spinal interneurons and their cell bodies are the target neurons and sites for the categories of oral and parenteral medications comprise the anticonvulsants, psychotropes including the antidepressants and the opioids. The neural sites and circuitries are the neural endings and receptors, axons, cell bodies, axon-axonal, axon-soma and dendrodendritic sites in the peripheral, ganglia, the spinal cord and brain. The effects infra of said medications are exerted on the neuronal receptors and endings, membranes and organelles of said neurons in the peripheral and central nervous systems. The effects include the modulation of releases and uptake of neurochemicals, neurohormones, the excitatory and inhibitory activities and the action potential thresholds of said neurons.

For example, in fibromyalgia, gabapentin or amitriptyline can exert its effect on the receptors and membrane of said small and medium unmyelinated and myelinated afferent neurons, postganglionic sympathetic neurons, preganglionic sympathetic neurons and spinal interneurons in the peripheral and central nervous systems. The effect may be excitatory or inhibitory depending on said types of neurons being affected. Therefore, the activities of the neural circuitry involving said neurons are affected or modulated.

Said categories of medications include the precursors of said medications, their synthetic derivatives produce in the laboratories and the metabolites of said medications form in a human body.

Said medications can be orally ingested, injected into the cardiovascular system or introduced into the body through the skin for the diffusion down a chemical gradient.

Although the preferred embodiments have been described for the categories of medications described herein, it will be appreciated by those skilled in pharmacology and medicine that other medications with similar pharmacologic and neurophysiological properties can be used without departing from the spirit of the invention and the scope of the claims.

1. The categories of oral medications for managing and treating fibromyalgia and related diseases, disorders, syndromes and sequelae in a human base on the anatomy and neurophysiology described in the Neurophysiologic Basis of Idiopathic Diseases adaptable to reach the target neurons involve in the genesis and perpetuation of fibromyalgia and related syndromes and sequelae in the peripheral nervous system and central nervous system comprises:
a category of medications affecting and modulating said target neurons;
a category of medications affecting the neural circuitry comprising said target neurons;
a category of medications affecting the organelles of said target neurons;
a category of medications affecting the neural membranes of said target neurons;
a category of medications affecting the receptors of said target neurons;
a category of medications affecting and modulating the action potentials of said target neurons;
a precursor of said medication; and
a derivative including the metabolites of said medication.

2. The categories of oral medication means according to claim 1 wherein said category of medications affecting said target neurons comprises the anticonvulsants.

3. The anticonvulsants according to claim 2 include gamma-aminobutyric acid analogues including tiagabine and gabapentin, benzodiazepines, carbamazepine, clonazepam, hydantoins, phenytoin, baclofen, valproate, valproic acid, barbiturates, topiramate, oxcabazepine, zonisamide, divalproex, felbamate, levetiracetam, succinimides and phenyltriazines.

4. The categories of oral medication means according to claim 1 wherein said category of medications affecting said target neurons comprises the psychotropes including the antidepressants.

5. The antidepressants according to claim 4 include amitriptyline, nortriptyline, protriptyline, imipramine, marpotiline, paroxetine, fluoxetine, sertraline, mirtazapine, nefazodone, bupropion, venlafaxine, desipramine, clomipramine, citalopram and doxepin.

6. The categories of oral medication means according to claim 1 wherein said category of medications affecting and modulating said target neurons comprises the opioids.

7. The categories of oral medication means according to claim 1 wherein said category of medications affecting said target neurons is the anticonvulsants.

8. The categories of oral medication means according to claim 1 wherein said category of medications affecting said target neurons is the psychotropes including the antidepressants.

9. The categories of oral medication means according to claim 1 wherein said category of medications affecting the neural circuitry comprising said target neurons is the anticonvulsants.

10. The categories of oral medication means according to claim 1 wherein said category of medications affecting the neural circuitry comprising said target neurons is the psychotropes including the antidepressants.

11. The categories of oral medication means according to claim 1 wherein said category of medications affecting the organelles of said target neurons is the anticonvulsants.

12. The categories of oral medication means according to claim 1 wherein said category of medications affecting the organelles of said target neurons is the psychotropes including the antidepressants.

13. The categories of oral medication means according to claim 1 wherein said category of medications affecting the receptors of said target neurons is the anticonvulsants.

14. The categories of oral medication means according to claim 1 wherein said category of medications affecting the receptors of said target neurons is the psychotropes including the antidepressants.

15. The categories of oral medication means according to claim 1 wherein said category of medications affecting the neural membranes of said target neurons is the anticonvulsants.

16. The categories of oral medication means according to claim 1 wherein said category of medications affecting the neural membranes of said target neurons is the psychotropes including the antidepressants.

17. The categories of oral medication means according to claim 1 wherein said category of medications affecting and modulating the action potentials of said target neurons is the anticonvulsants.

18. The categories of oral medication means according to claim 1 wherein said category of medications affecting and modulating the action potentials of said target neurons is the psychotropes including the antidepressants.

19. The categories of oral medications for managing and treating fibromyalgia and related diseases, disorders, syndromes and sequelae in a human base on the anatomy and neurophysiology described in the Neurophysiologic Basis of Idiopathic Diseases adaptable to affect and modulate the cellular activities and organelles including the membranes, ions channels and the release of neurochemicals and neurohumorals of the target neurons involve in the genesis and perpetuation of fibromyalgia and related diseases and disorders in the peripheral nervous system and central nervous system comprises:
a category of medications affecting and modulating said target neurons;
a category of medications affecting the neural circuitry comprising said target neurons;
a category of medications affecting the organelles of said target neurons;
a category of medications affecting the neural membranes of said target neurons;
a category of medications affecting the receptors of said target neurons;
a category of medications affecting and modulating the action potentials of said target neurons;
a precursor of said medication; and
a derivative including the metabolites of said medication.

20. The categories of parenteral medications for managing and treating fibromyalgia and related diseases, disorders, syndromes and sequelae in a human base on the anatomy and neurophysiology described in the Neurophysiologic Basis of Idiopathic Diseases adaptable to affect and modulate the cellular activities and organelles including the membranes, ions channels and the release of neurochemicals and neurohumorals of the target neurons involve in the genesis and perpetuation of fibromyalgia and related diseases and disorders in the peripheral nervous system and central nervous system comprises:
a category of medications affecting and modulating said target neurons;
a category of medications affecting the neural circuitry comprising said target neurons;
a category of medications affecting the organelles of said target neurons;
a category of medications affecting the neural membranes of said target neurons;
a category of medications affecting the receptors of said target neurons;
a category of medications affecting and modulating the action potentials of said target neurons;
a precursor of said medication; and
a derivative including the metabolites of said medication.
